(19) United States
(12) Patent Application Publication
(10) Pub. No.: US 2024/0358747 A1
(43) Pub. Date: Oct. 31, 2024

(54) SOME GEL FORMULATIONS THAT PROVIDE WOUND HEALING AND THE SYNTHESIS OF DYSPROSIUM ERBIUM BORATE TRIHYDRATE (Dy0.5Er0.5BO3.3H2O) COMPOUND USED IN THESE FORMULATIONS

(71) Applicant: VERTRATECH KIMYA SANAYI VE TICARET LIMITED SIRKETI, Bigadiç, Balikesir (TR)

(72) Inventor: Ayse Ecem UYSAL, Bigadiç, Balikesir (TR)

(21) Appl. No.: 18/687,936

(22) PCT Filed: Aug. 17, 2022
(86) PCT No.: PCT/TR2022/050872

§ 371 Date: Feb. 29, 2024

(30) Foreign Application Priority Data
Sep. 1, 2021 (TR) 2021/013738

(51) Int. Cl. A61K 33/244; A61K 9/00; A61K 9/06; A61K 31/728; A61K 47/32

(52) U.S. Cl. CPC A61K 33/244; A61K 9/0014; A61K 9/06; A61K 31/728; A61K 47/32

(57) ABSTRACT

Disclosed is a gel formulation, in which boric acid, metal borate and hyaluronic acid derivatives are used together as an active ingredient, which is rapidly absorbed from the skin, effectively accelerating the skin's self-renewal, removal of redness and scar-free wound healing processes. Additionally disclosed is the synthesis of a novel amorphous, nanostructured bimetallic compound of dysprosium erbium borate trihydrate used in this formulation.

### FIELD OF THE INVENTION

The present invention relates to a gel formulation, in which boric acid, metal borate and hyaluronic acid derivatives are used together as an active ingredient, which is rapidly absorbed from the skin, effectively accelerating the skin's self-renewal, removal of redness and scar-free wound healing processes and to the synthesis of a novel amorphous, nanostructured bimetallic compound of dysprosium erbium borate trihydrate used in this formulation.

### STATE OF THE ART

Wound is the temporary or complete loss of physiological properties due to the deterioration of tissue integrity of the skin or mucosa due to different reasons such as biological, physical, chemical and radiation (1, 2). The morphology and characteristics of the wound vary according to the cause of the wound. At the same time, factors such as the age of the individual and the health status of the individual also affect the morphology of the wound and the healing of the wound (3).

Wounds are divided into acute or chronic according to their type and etiology. Acute wounds are; lacerations, burns, and surgical incisions. Chronic wounds are; pressure sores, diabetic ulcers, venous ulcers and arterial ulcers (4).

Acute wounds are wounds that are caused by a temporary factor and in which tissue can return to its normal anatomical and functional integrity in an acceptable time. There are few factors that prevent the healing of these wounds. Chronic wounds, on the other hand, are non-healing or slow-healing (longer than three months) wounds caused by a persistent factor, unlike acute wounds. Even a simple physical trauma can cause a chronic wound that cannot be healed (5).

Wound healing is a complex process consisting of the integration of physiological and biochemical events, a large number of cells, cytokines or mediators produced by the cells, and the extracellular matrix, all wounds heal with the same basic principles. In cases where skin integrity is impaired, a process begins wherein many cells including keratinocytes, fibroblasts, endothelium, macrophages and platelets are involved. New tissue structure is formed with the migration, spread, proliferation and differentiation of these cells and thus the wound is closed. Growth hormones, cytokines and chemokines play a role in this process. In particular, immunohistochemical evaluation of collagen, cytokines and growth factors is important for determining the wound healing process (6).

The area of the wound and factors such as blood flow, cytokines and growth factors, genetic and immunological disorders, diabetes, radiotherapy, chemotherapy, improper nutrition, and steroid drug use affect wound healing. Healthy wound healing is ensured by sufficient tissue perfusion, oxygenation and epithelialization, as well as good nutrition and moistening of the tissue (7).

Hyaluronic acid (Hyaluronan, HA) is a naturally occurring substance in all living organisms, from the simplest bacteria to the most advanced. Hyaluronic acid (HA), a member of the hyaluronan family, was first discovered in 1934 by K. Meyer and John W. Palmer (8).

Hyaluronic acid (HA) is a nitrogen-containing mucopolysaccharide composed of D-glucuronic acid-N-acetylglucosamine disaccharide units and is an important component of the extracellular matrix. HA which is an anionic glucosaminoglycan (GAG) with a high water holding capacity, can reach a molecular weight of 107 Da. It has a wide application area in bioengineering and biomedicine due to its high water retention capacity, viscoelastic structure, high biocompatibility and not forming toxic components during degradation (9, 10).

It is known that the biological activities of hyaluronic acid differ depending on the molecular weight. For example, hyaluronic acid with a molecular weight of 0.4 to 4.0 kDa acts as an inducer of heat shock proteins and has non-apoptotic feature. Polysaccharides with a molecular weight equal to 6-20 kDa have immunostimulating, angiogenic and phlogotic activities. Hyaluronic acid with a molecular weight of 20-200 kDa is involved in biological processes such as embryonic development, wound healing and ovulation. On the contrary, high molecular weight hyaluronic acid (>500 kDa) has anti-angiogenic activity and may act as a space-filler and a natural immunological depressant (8).

HA acts as a biologically active molecule that regulates the tissue repair process and is considered a safe and effective option for use in skin repair. It is observed that the presence of hyaluronic acid at the wound area increases shortly after a wound is formed. This is due to the two vitally important functions that HA serve in the wound healing process. In the early stages of wound healing, hyaluronic acid mainly acts as a temporary structure due to its large molecular size. Its unique structure facilitates the diffusion of nutrients and waste products from the injury site. Even more important is the interaction of hyaluronic acid with keratinocytes. Keratinocytes are cells in the epidermis of the skin who serves to produce keratin and form tight connections. HA directly affects the proliferation and migration of these cell types (11).

Irregularity of HA metabolism, which is commonly found in connective tissues and is known to be an integral part of the repair process, causes non-healing and hard-to-heal wounds. Decreased rate of biosynthesis of extracellular matrix components, especially HA, in human chronic dermal wounds and diabetic injured skin causes a significant delay in collagen tissue formation and angiogenesis. As a result of the researches, when hyaluronic acid is applied systematically to the wound area, It is seen that it increases collagen production and also contributes to the formation of new vessels in the wound area, accelerating the healing process and effectively treating diabetic wounds (12).

Many applications have been made in the state of the art regarding the wound healing activity of hyaluronic acid. In the patent application numbered EP3191133A1, which is one of these applications, hyaluronic acid or its derivatives, a borate compound as crosslinker (for example, boric acid, sodium tetraborate, tributyl borate, etc.), a di or polyvalent metal ion (Ca⁺², Sr⁺², Cd⁺², Cu⁺², Mg⁺² etc.) and optionally one or more cationic monomers (polyethyleneimine, hydroxylysine, etc.) are used to form pharmaceutical formulations in different combinations. It is reported that the composition disclosed in this application can be used in the repair of cuts, tears, bone fractures and other injuries resulting from surgery or trauma.

Methods for inhibiting scar formation in the wound area are described in the patent application numbered US20070059377A1. An alkaline pH formulation was created by using hyaluronic acid and alkalizing agent (sodium bicarbonate, sodium hydroxide and potassium hydroxide, etc.) together to reduce scar formation in the wound area. It is stated that this formulation also prevents scar formation by helping the wound area to heal.

The patent application numbered U.S. Pat. No. 9,265,793B2 relates to the creation of a pharmaceutical formulation consisting of hyaluronic acid with an average molecular weight between 130 and 230 KDa and silver in colloidal metallic form or in a micronized metallic form like a porous sponge. The composition described herein is mentioned to promote healing of various types of lesions (acute, chronic, ulcerations of various etiology, burns, wounds), eliminate bacterial and/or fungal infections and promote the healing of damaged tissue.

The atomic number of boron, represented by the symbol B in the periodic table, is 5 and its atomic weight is 10.81. Boron element, which has semi-metal and semiconductor properties, is in group 3A of the periodic table. Boron, as an element, consists of two separate stable isotopes called B10 and B111 in nature. The element boron, which is never found in free form in nature, forms compounds with different properties with various metal or nonmetal elements (13).

Negatively charged ion radicals in which the boron atom is in triple or quadruple coordination with the oxygen atom are called borates, borate compounds can only be formed from plane triangular (BO₃)⁻³ or tetrahedral (BO₄)⁻⁵ anions, or they can be composed of mixtures of both (BO₃)⁻³ and (BO₄)⁻⁵ groups.

Rare earth metals (REE) (Lanthanides) are an extremely important and strategic raw material source used in advanced technology due to their characteristic high melting point, conductivity and heat permeability, as well as being highly resistant to external factors such as abrasion and corrosion. Lanthanide borate compounds (LnₓB_{y}O_{z}/LnₓB_{y}O_{z}Hₜ), which are formed by the combination of rare earth metal ions and borate anions, have nonlinear optical (NLR) properties, thermal stability, high optical damage threshold, ultraviolet light transmittance and strong luminescence character. Due to these properties, monodisperse nano-lanthanide borates are very important for applications in materials and medicine, especially with their paramagnetic and optical properties. In many studies to date, it has been reported that boron-containing compounds are effective in the wound healing process by stimulating growth factor release, increasing the extracellular matrix turnover, inducing vascularization and immunomodulation (14). It has been observed that topical application of 3% boric acid to wounds reduces the length of stay in intensive care units three times. In another study, diabetic animals have been proven to increase wound healing rate and histopathological scores, when the gel formulation containing sodium pentaborate was evaluated in terms of its antimicrobial activities, proliferation, angiogenesis, gene and growth factor expression promoting effects on dermal cells in vitro (15).

In the last few years, there are new and exciting studies on the wound healing effect of nano-sized lanthanide borate compounds in the literature. For example, in a study, wound healing activities of sodium pentaborate pentahydrate (NaB), pluronic (Plu; F68 and F127) and their combinations were investigated using in vitro and in vivo approaches. As a result, formulations prepared using sodium pentaborate pentahydrate (NaB), pluronic (Plu; F68 and F127) have been shown to increase fibroblast migration, antioxidant enzyme activity, and acute wound cutaneous wound healing (16).

In the patent application numbered TR 2018 16274, the accelerating effects of erbium borate nanoparticles on the wound healing process were examined and it was seen that scar-free wound formation was achieved. In the same patent application, it was shown that monometallic and nano-sized dysprosium borate (D_{y}BO₃·3H₂O) compound increases cell viability of HaCaT cells and accelerates the wound healing process by stimulating wound healing genes.

In the state of the art, it seems that recent research on skin injury and wound healing mainly focuses on infection prevention, dermal regeneration and angiogenesis. In addition to this, it was determined that less attention was paid to air permeability and prevention of moisture loss in studies. However, these factors play important roles in the healing of wounds. After all, several problems with the healing of skin wounds are not fully solved. One of them is that hemostatic and anti-infective materials are generally ineffective in promoting wound healing, and another is that growth factors can induce hyperblastosis or tumorigenesis. The failure of the active ingredients used for wound healing in the studies in the literature to increase the rate of cell division causes scar formation in the wound area, which causes aesthetic anxiety for most people. Besides, pentaborate is a compound that needs the synthesis of sodium pentaborate compound used in gel formulation containing pluronic and their combination. This causes a large increase in the cost of the gel.

As a result, due to the abovementioned disadvantages, deficiencies, there is a requirement to make an innovation in the relevant technical field.

### Aim of the Invention

The present invention relates to some gel formulations that provide wound healing and the synthesis of dysprosium erbium borate trihydrate (Dy_{0.5}Er_{0.5}BO₃·3H₂O) compound used in these formulations that meets the above-mentioned requirements, eliminates all disadvantages and brings some additional advantages.

The main aim of the present invention is to obtain a gel formulation, in which boric acid, metal borate and hyaluronic acid derivatives are used together as an active ingredient, which is rapidly absorbed from the skin, effectively accelerating the skin's self-renewal, removal of redness and scar-free wound healing processes

The aim of the invention is to synthesize borate derivatives with superior wound healing properties by adjusting boric acid to the appropriate pH range with sodium hydroxide solution.

The aim of the invention is to introduce a new borate derivative with superior properties in wound healing compared to monometallic dysprosium borate and erbium borate compounds.

The aim of the invention is to reveal an amorphous, nanostructured and bimetallic borate derivative with the chemical formula Dy_{0.5}Er_{0.5}BO₃·3H₂O.

The aim of the invention is to synthesize a new amorphous, nano-structured and bimetallic Dy_{0.5}Er_{0.5}BO₃·3H₂O (dysprosium erbium borate trihydrate) compound by using the co-precipitation method, which is convenient for fabrication, easy and economical compared to the solid state synthesis method and to prepare stable colloidal solutions of this compound.

The aim of the invention is to provide a low-cost gel formulation with an easy production process so as to achieve a scar-free healing.

In order to fulfill the above mentioned aims, the present invention is an amorphous, nanostructured and bimetallic borate derivative with the chemical formula Dy_{0.5}Er_{0.5}BO₃·3H₂O.

In order to realize the objectives of the invention, amorphous, nanostructured and bimetallic borate derivative with the chemical formula Dy_{0.5}Er_{0.5}BO₃·3H₂O is synthesized by the following process steps;
dissolving the weighed erbium nitrate and dysprosium nitrate in 20 ml of water in a 250 ml beaker to obtain a metal nitrate solution,
dissolving the weighed PEG 2000 in 180 ml of water in a 250 ml beaker to obtain the PEG solution,
adding the PEG solution to the metal nitrate solution and mixing for 30 minutes to obtain the PEG-metal nitrate mixture,
dissolving the weighed sodium hydroxide in 100 ml of water and adding boric acid to it and mixing for 10 minutes so as to prepare a borate buffer solution,
mixing the PEG-metal nitrate mixture at 2000 rpm in a mechanical mixer,
adding the prepared borate buffer solution to the PEG-metal nitrate mixture during mixing, and mixing at the same speed for 30 minutes,
measuring the pH value of the mixture by pouring the milky mixture obtained into a large beaker,
centrifuging the mixture obtained at 7000 rpm for 5 minutes by dividing the same into 12 centrifuge tubes and discarding the formed filtrate,
washing the remaining solid part by shaking it thoroughly with pure water for the first time with the help of a vortex, centrifuging at 7000 rpm for 5 minutes and discarding the resulting filtrate,
washing the solid part with pure water a second time with the help of a vortex again, centrifuging the same at 7000 rpm for 5 minutes and discarding the resulting filtrate,
washing the solid part with pure water a third time with the help of a vortex again, centrifuging the same at 7000 rpm for 30 minutes and collecting the filtrate in the form of colloidal dispersion obtained in a glass jar.

In order to fulfil the above-described purposes, the invention is a gel formulation production method that absorbs quickly from the skin, renews the skin, eliminates redness, and accelerates the wound healing process effectively, resulting in a scar-free healing, characterized by comprising the following process steps;
dispersing the gelling agent in the water in the beaker,
adding hyaluronic acid and boric acid with a molecular weight of 500 KDa or less on this mixture, and mixing the same until complete dissolution,
adding 20% NaOH solution to the reaction medium to ensure borate formation and gelation and adjusting the medium to the appropriate pH value,
keeping the resulting mixture at 4° C. for approximately 24 hours.

In order to fulfil the above-described purposes, the invention is a gel formulation production method that absorbs quickly from the skin, renews the skin, eliminates redness, and accelerates the wound healing process effectively, resulting in a scar-free healing, characterized by comprising the following process steps;
dispersing the gelling agent in the water in the beaker,
adding hyaluronic acid and boric acid with a molecular weight of 500 KDa or less on this mixture, and mixing the same until complete dissolution,
adding 20% NaOH solution to the reaction medium to ensure borate formation and gelation and adjusting the medium to the appropriate pH value,
adding dysprosium erbium borate trihydrate to the prepared gel,
keeping the resulting mixture at 4° C. for approximately 24 hours.

In order to fulfil the above-described purposes, the invention is a gel formulation production method that absorbs quickly from the skin, renews the skin, eliminates redness, and accelerates the wound healing process effectively, resulting in a scar-free healing, characterized by comprising the following process steps;
dispersing the gelling agent in the water in the beaker,
adding hyaluronic acid with a molecular weight of 500 KDa or less on this mixture, and mixing the same until complete dissolution,
adding 20% NaOH solution to the reaction medium to ensure borate formation and gelation and adjusting the medium to the appropriate pH value,
adding dysprosium erbium borate trihydrate to the prepared gel,
keeping the resulting mixture at 4° C. for approximately 24 hours.

The structural and characteristic features of the present invention will be understood clearly by the following detailed description and therefore the evaluation shall be made by taking the detailed description into consideration.

### DETAILED DESCRIPTION OF THE INVENTION

In this detailed description, the invention, some gel formulations that provide wound healing and the synthesis of dysprosium erbium borate trihydrate (Dy_{0.5}Er_{0.5}BO₃·3H₂O) compound used in these formulations is described only for clarifying the subject matter in a manner such that no limiting effect is created.

The present invention relates to a gel formulation, in which boric acid, metal borate and hyaluronic acid derivatives are used together as an active ingredient, which is rapidly absorbed from the skin, effectively accelerating the skin's self-renewal, removal of redness and scar-free wound healing processes. and to the synthesis of a novel amorphous, nanostructured bimetallic compound of dysprosium erbium borate trihydrate used in this formulation.

### The Formulation of the Dysprosium Erbium Borate Trihydrate Compound Used in the Inventive Formulation:

| | Substance | Substance | Solvent | |
|---|---|---|---|---|
| | amount | amount | amount | Usable amount |
| Content | (mmol) | (g) | (ml) | by weight (%) |
| Erbium Nitrate | 5 | 2.2167 | | 0.1-1.0 |
| Dysprosium Nitrate | 5 | 2.1929 | | 0.1-1.0 |
| Sodium hydroxide | 45 | 1.8000 | | 0.1-1.0 |
| Boric Acid | 90 | 5.5649 | | 1.0-2.0 |
| PEG 2000 | 20 | 40.0000 | | 10-40 |
| Water | | | 300 | 88.7-57.7 |

### Synthesis of Dv_{0.5}Er_{0.5}BO₃·3H₂O (Dysprosium Erbium Borate Trihydrate) and Preparation Method of Stable Colloidal Dispersions

The weighed erbium nitrate and dysprosium nitrate substances are dissolved in 20 ml of water in a 250 ml beaker to obtain a metal nitrate solution,
The weighed PEG 2000 is dissolved in 180 ml of water in a 250 ml beaker to obtain the PEG solution,
The PEG solution is added to the metal nitrate solution and PEG-metal nitrate mixture is obtained by mixing for 30 minutes,
The weighed sodium hydroxide is dissolved in 100 ml of water and boric acid is added to it and mixed for 10 minutes so as to prepare a borate buffer solution,
The PEG-metal nitrate mixture is taken into a three-necked flask and placed in the mechanical mixer assembly and mixed at a mixing speed of 2000 rpm,
During mixing, the prepared borate buffer solution is added to the three-necked reaction flask containing the PEG-metal nitrate mixture and mixed at this speed for 30 minutes,
The pH value of the mixture is measured by pouring the milky mixture obtained into a large beaker,

(At this stage, the pH value of the milky mixture is 8-9.)
The resulting mixture is divided into 12 centrifuge tubes, centrifuged at 7000 rpm for 5 minutes and the resulting filtrate is discarded,
The remaining solid part is washed with pure water for the first time with the help of a vortex, it is centrifuged at 7000 rpm for 5 minutes and the resulting filtrate is discarded,
The solid part is washed with pure water a second time with the help of a vortex again, it is centrifuged at 7000 rpm for 5 minutes and the resulting filtrate is discarded,
The solid part is washed with pure water for the third time with the help of a vortex again, centrifuged at 7000 rpm for 30 minutes and the filtrate in the form of colloidal dispersion is collected in a glass jar. This colloidal dispersion obtained has a very high stability and can remain stable for months without precipitation.

The inventive amorphous, nano-structured and bimetallic Dy_{0.5}Er_{0.5}BO₃·3H₂O (dysprosium erbium borate trihydrate) compound is synthesized by using the co-precipitation method, which is convenient for fabrication, easy and economical compared to the solid state synthesis method.

A plurality of gel formulations were obtained by using boric acid and different borate derivatives (molecular or nanostructured) and hyaluronic acid derivatives below 500 KDa in order to achieve a scar-free healing on the applied skin within the scope of the invention.

Formulation 1

| | | Amount used |
|---|---|---|
| | | by weight |
| Content | Function | (w/w %) |
| Boric Acid (H₃BO₃) | Active/Protective | 1.0-5.0 |
| Dysprosium Erbium Borate | Active | 0.1-1.0 |
| Trihydrate (Dy_{0.5}Er_{0.5}BO₃•3H₂O) | | |
| Hyaluronic acid with a molecular | Active | 0.1-5.0 |
| weight of 500 KDa or less | | |
| Tween −20 etc. | Surfactant | 1.0-5.0 |
| Chlorhexidine etc. | Protective | 0.1-2.0 |
| Carbomer etc. | Gelling agent | 0.1-1.0 |
| 20% NaOH solution | Neutralizer | 0.1-3.0 |
| Water | Solvent | 97.5-78.0 |

The gelling agent (0.1-1.0 g) is weighed and dispersed in water (97.5-78.0 mL) in a 250 ml beaker. Hyaluronic acid (0.1-5.0 g) and boric acid (1.0-5.0 g) with a molecular weight of 500 KDa or less are added to this mixture and mixed until complete dissolution is achieved. Then, 20% NaOH solution (0.1-3.0 g) is added to the medium to ensure borate formation and gelation, and the medium is adjusted to the appropriate pH value. Dysprosium erbium borate trihydrate (Dy_{0.5}Er_{0.5}BO₃·3H₂O) solution (0.1-1.0 mL) is added into the prepared gel. Surfactant (1.0-5.0 g) and preservative (0.1-2.0) are added to the gel obtained in the last step. The mixture is kept at 4° C. for approximately 24 hours.

In the preferred embodiment of the invention, the pH is adjusted to the range of 6-7.5.

In the preferred embodiment of the invention, carbomer or the like can be used as a gelling agent, Tween-20 or the like as a surfactant, and chlorhexidine or the like can be used as a preservative.

Formulation 2

| | | Amount used |
|---|---|---|
| | | by weight |
| Content | Function | (w/w %) |
| Boric Acid (H₃BO₃) | Active/Protective | 1.0-5.0 |
| Dysprosium Erbium Borate Trihydrate | Active | 0.1-1.0 |
| (Dy_{0.5}Er_{0.5}BO₃•3H₂O) | | |
| Hyaluronic acid with a molecular | Active | 0.1-5.0 |
| weight of 500 KDa or less | | |
| Carbomer etc. | Gelling agent | 0.1-1.0 |
| 20% NaOH solution | Neutralizer | 0.1-3.0 |
| Water | Solvent | 98.6-85.0 |

The gelling agent (0.1-1.0 g) is weighed and dispersed in water (98.6-85.0 mL) in a 250 ml beaker. Hyaluronic acid (0.1-5.0 g) and boric acid (1.0-5.0 g) with a molecular weight of 500 KDa or less are added to this mixture and mixed until complete dissolution is achieved. Then, 20% NaOH solution (0.1-3.0 g) is added to the medium to ensure borate formation and gelation, and the medium is adjusted to the appropriate pH value. Dysprosium erbium borate trihydrate (Dy_{0.5}Er_{0.5}BO₃·3H₂O) solution (0.1-1.0 mL) is added into the prepared gel. The mixture is kept at 4° C. for approximately 24 hours.

In the preferred embodiment of the invention, the pH is adjusted to the range of 6-7.5.

In the preferred embodiment of the invention, carbomer or the like can be used as gelling agent.

Formulation 3

| | | Amount used |
|---|---|---|
| | | by weight |
| Content | Function | (w/w %) |
| Dysprosium Erbium Borate Trihydrate | Active | 0.1-1.0 |
| (Dy_{0.5}Er_{0.5}BO₃•3H₂O) | | |
| Hyaluronic acid with a molecular | Active | 0.1-5.0 |
| weight of 500 KDa or less | | |
| Tween −20 etc. | Surfactant | 1.0-5.0 |
| Chlorhexidine etc. | Protective | 0.1-2.0 |
| Carbomer etc. | Gelling agent | 0.1-2.0 |
| 20% NaOH solution | Neutralizer | 0.1-3.0 |
| Water | Solvent | 98.5-83.0 |

The gelling agent (0.1-1.0 g) is weighed and dispersed in water (98.5-84.0 mL) in a 250 ml beaker. Hyaluronic acid (0.1-5.0 g) with a molecular weight of 500 KDa or less are added to this mixture and mixed until complete dissolution is achieved. 20% NaOH solution (0.1-3.0 g) is added to the medium to ensure the gelation of the carbomer and to neutralize the pH of the medium. Dysprosium erbium borate trihydrate (Dy_{0.5}Er_{0.51}BO₃·3H₂O) solution (0.1-1.0 mL) is added into the prepared gel. Surfactant (1.0-5.0 g) and preservative (0.1-2.0) are added onto the mixture obtained. The mixture is kept at 4° C. for approximately 24 hours.

In the preferred embodiment of the invention, the pH is adjusted to the range of 6-7.5.

In the preferred embodiment of the invention, carbomer or the like can be used as a gelling agent, Tween-20 or the like as a surfactant, and chlorhexidine or the like can be used as a preservative.

Formulation 4

| | | Amount used |
|---|---|---|
| | | by weight |
| Content | Function | (w/w %) |
| Boric Acid (H₃BO₃) | Active/Protective | 1.0-5.0 |
| Hyaluronic acid with a molecular | Active | 0.1-5.0 |
| weight of 500 KDa or less | | |
| Tween −20 etc. | Surfactant | 1.0-5.0 |
| Chlorhexidine etc. | Protective | 0.1-2.0 |
| Carbomer etc. | Gelling agent | 0.1-1.0 |
| 20% NaOH solution | Neutralizer | 0.1-3.0 |
| Water | Solvent | 97.6-79.0 |

The gelling agent (0.1-1.0 g) is weighed and dispersed in water (97.6-80.0 mL) in a 250 ml beaker. Hyaluronic acid (0.1-5.0 g) and boric acid (1.0-5.0 g) with a molecular weight of 500 KDa or less are added to this mixture and mixed until complete dissolution is achieved. Then, 20% NaOH solution (0.1-3.0 g) is added to the medium to ensure borate formation and gelation, and the medium is adjusted to the appropriate pH value. Surfactant (1.0-5.0 g) and preservative (0.1-2.0) are added to the gel obtained in the last step. The mixture is kept at 4° C. for approximately 24 hours.

In the preferred embodiment of the invention, the pH is adjusted to the range of 6-7.5.

In the preferred embodiment of the invention, carbomer or the like can be used as a gelling agent, Tween-20 or the like as a surfactant, and chlorhexidine or the like can be used as a preservative.

Formulation 5

| | | Amount used |
|---|---|---|
| | | by weight |
| Content | Function | (w/w %) |
| Boric Acid (H₃BO₃) | Active/Protective | 1.0-5.0 |
| Hyaluronic acid with a molecular | Active | 0.1-5.0 |
| weight of 500 KDa or less | | |
| Carbomer etc. | Gelling agent | 0.1-1.0 |
| 20% NaOH solution | Neutralizer | 0.1-3.0 |
| Water | Solvent | 98.7-86.0 |

The gelling agent (0.1-1.0 g) is weighed and dispersed in water (98.7-86.0 mL) in a 250 ml beaker. Hyaluronic acid (0.1-5.0 g) and boric acid (1.0-5.0 g) with a molecular weight of 500 KDa or less are added to this mixture and mixed until complete dissolution is achieved. Then, 20% NaOH solution (0.1-3.0 g) is added to the medium to ensure borate formation and gelation, and the medium is adjusted to the appropriate pH value. The mixture is kept at 4° C. for approximately 24 hours.

In the preferred embodiment of the invention, the pH is adjusted to the range of 6-7.5. In the preferred embodiment of the invention, carbomer or the like can be used as gelling agent.

Boric acid, which is used as an active ingredient in the gel formulations set forth within the scope of the invention, was converted into borate derivatives by selecting the appropriate pH value with sodium hydroxide solution, without the need for an additional synthesis process. In addition, an amorphous, bimetallic and nano-structured Dy0.5Er0.5BO3.3H2O compound that was never synthesized before was synthesized and used as an active ingredient in our wound healing formulations.

This new bimetallic and nanostructured Dy0.5Er0.5BO3.3H2O compound obtained has much superior properties in wound healing compared to monometallic dysprosium borate and erbium borate compounds. The use of boric acid, metal borate derivatives and hyaluronic acid together as an active ingredient has created a superior synergistic effect and this effect has made the wound healing gel superior and advantageous over the existing gels used for wound healing.

### REFERENCES

1. Phillips L G. 1998. Wound Healing. In: Alberts B. Surgical Basic Principles. United States of America: Garland Inc. 131-143.
2. Boateng, J. S. Matthews, K. H. Stevens, H. N. ve Eccleston, G. M. 2008. Wound healing dressings and drug delivery systems: a review, Journal of pharmaceutical sciences. 97: 2892-2923.
3. Kurto\check{g}lu, A. H., ve Karataş A. 2009. Yara tedavisinde güncel yaklaşimlar: modern yara örtüleri. Ankara Üniversitesi Eczacilik Fakültesi Dergisi. 38 (3): 211-232.
4. Çiçek M. 2015. Deneysel Olarak Oluşturulan Yara Modelinde Akupunktur'un Yara Iyileşmesi Üzerine Etkisinin Araştirilmasi. Yüksek Lisans Tezi. Tokat: Gaziosmanpaşa Üniversitesi Sa\check{g}lik Bilimler Enstitüsü.
5. Baktir G. 2019. Yara Iyileşmesi ve Deneysel Yara Modelleri. Istanbul Yeni Yüzyil Üniversitesi Eczacilik Fakültesi, Farmakoloji Anabilim Dali. 9(3): 130-7.
6. Singer A J, Clark R A. 1999. Cutaneous wound healing. N Engl J Med: 341:738-46.
7. Rajpaul K. 2015. Biofilm in wound care. Sup¬pl Wound Care: S6, S8, S10-1.
8. Snetkov, P, Zakharova K. ve ark. 2020. Hyaluronic acid The Influence of Molecular Weight on Structural, Physical, Physico-Chemical, and Degradable Properties of Biopolymer. 12 (8): 1800.
9. Yan S, Zhang Q, Wang J. ve ark. 2013. Silk fibroin/chondroitin sulfate/hyaluronic acid ternary scaffolds for dermal tissue reconstruction. 9: 6771-6782.
10. Meinert C, Schrobback K. ve ark. 2017. A novel bioreactor system for biaxial mechanical loading enhances the properties of tissue-engineered human cartilage. Bilim Cum: 7:16997.
11. Jeffrey Voigt, Vickie R. Driver. 2012. Hyaluronic acid derivatives and their healing effect on burns, epithelial surgical wounds, and chronic wounds: a systematic review and meta-analysis of randomized controlled trials. 20 (3):317-331.
12. Galeano M., Polito F., Bitto A., Irrera N. ve ark. 2011. Systemic administration of high-molecular weight hyaluronan stimulates wound healing in genetically diabetic mice. 7:752-759.
13. Yakinci Z D, Kök M. 2016. Borun sa\check{g}lik alaninda kullanimi. T. C. Inönü Üniversitesi Sa\check{g}lik Hizmetleri Meslek Yüksekokulu Dergisi. 4(1):36-44.
14. Bozkurt B., Taşli P. N., Koçak P., Bülbül B. ve ark. 2019. Uluslararasi Bor Sempozyumu. 17-19 Nisan 2019 Nevşehir: Haci Bektaş Veli Üniversitesi Kültür ve Kongre Merkezi.
15. Demirci S., Do\check{g}an A., Aydin S., Çikler E.ve ark. 2016. Boron promotes streptozotocin-induced diabetic wound healing: roles in cell proliferation and migration, growth factor expression, and inflammation. 417:119-113.
16. Do\check{g}an A., Demirci S., Ça\check{g}layan A. ve ark. 2014. Sodium Pentaborate Pentahydrate and Pluronic Containing Hydrogel Increases Cutaneous Wound Healing In Vitro and In Vivo. 162:72-79.

1. Amorphous, nanostructured and bimetallic borate derivative with the chemical formula Dy_{0.5}Er_{0.5}BO₃·3H₂O.

2. (canceled)

3. (canceled)

4. (canceled)

5. (canceled)

6. (canceled)

7. A gel formulation production method that absorbs quickly from the skin, renews the skin, eliminates redness, and accelerates the wound healing process effectively, resulting in a scar-free healing, the production method comprising the following process steps:
a) dispersing a gelling agent in water in a beaker,
b) adding hyaluronic acid and boric acid with a molecular weight of 500 KDa or less on this mixture, and mixing the same until complete dissolution,
c) adding 20% NaOH solution to the reaction medium to ensure borate formation and gelation and adjusting the medium to the appropriate pH value, and
d) keeping the resulting mixture at 4° C. for approximately 24 hours.

8. Gel formulation production method according to claim 7, comprising by weight 0.1-1.0% gelling agent, 98.7-79.0% or 98.7-86.0% water, 0.1-5.0% hyaluronic acid with a molecular weight of 500 KDa or less, 1.0-5.0% boric acid and 1.0-3.0% 20% NaOH.

9. Gel formulation production method according to claim 7, wherein said gelling agent is a carbomer or the like.

10. Gel formulation production method according to claim 7, comprising the process step of adding surfactant and preservative to the gel obtained after the c process step.

11. (canceled)

12. Gel formulation production method according to claim 10, comprising 1.5-5.0% surfactant and 0.1-2.0% preservative by weight.

13. Gel formulation production method according to claim 7, wherein said medium pH value is 6-7.5.

14. A gel formulation production method that absorbs quickly from the skin, renews the skin, eliminates redness, and accelerates the wound healing process effectively, resulting in a scar-free healing, comprising the following process steps:
a. dispersing a gelling agent in water in a beaker,
b. adding hyaluronic acid and boric acid with a molecular weight of 500 KDa or less on this mixture, and mixing the same until complete dissolution,
c. adding 20% NaOH solution to the reaction medium to ensure borate formation and gelation and adjusting the medium to the appropriate pH value,
d. adding dysprosium erbium borate trihydrate to the prepared gel, and
e. keeping the resulting mixture at 4° C. for approximately 24 hours.

15. Gel formulation production method according to claim 14, comprising, by weight 0.1-1.0% of gelling agent, 98.6-85.0% or 97.5-78.0% of water, 0.1-5.0% of hyaluronic acid with a molecular weight of 500 KDa or less, 1.0-5.0% boric acid, 0.1-1.0% dysprosium erbium borate trihydrate and 1.0-3.0% 20% NaOH solution.

16. Gel formulation production method according to claim 14, wherein said gelling agent is a carbomer or the like.

17. Gel formulation production method according to claim 21, comprising the process step of adding surfactant and preservative to the gel obtained after the d process step.

18. (canceled)

19. Gel formulation production method according to claim 17, comprising 1.5-5.0% surfactant and 0.1-2.0% preservative by weight.

20. Gel formulation production method according to claim 14, wherein said medium pH value is 6-7.5.

21. (canceled)

22. (canceled)

23. (canceled)

24. (canceled)

25. (canceled)

26. (canceled)

27. (canceled)
